(19) United States
(12) Patent Application Publication
(10) Pub. No.: US 2017/0087263 A1
(43) Pub. Date: Mar. 30, 2017

(54) ENDOSCOPE REPROCESSOR

(71) Applicant: OLYMPUS CORPORATION, Tokyo (JP)

(72) Inventor: Norito SATO, Sagamihara-shi (JP)

(73) Assignee: OLYMPUS CORPORATION, Tokyo (JP)

(21) Appl. No.: 15/377,310

(22) Filed: Dec. 13, 2016

Related U.S. Application Data
(63) Continuation of application No. PCT/JP2016/059917, filed on Mar. 28, 2016.

(30) Foreign Application Priority Data
Sep. 1, 2015 (JP) 2015-172204

(51) Int. Cl. A61L 2/18; B08B 9/032; B08B 11/00; B08B 3/08; B08B 3/10

(52) U.S. Cl. CPC A61L 2/18; B08B 3/08; B08B 3/10; B08B 11/00; B08B 9/0323; A61L 2202/24; B08B 2203/007; B08B 2209/032

(57) ABSTRACT

An endoscope reprocessor includes: a liquid supply nozzle including a lifting conduit configured to feed a liquid in a direction against gravity and a turndown conduit; a liquid ejection port; a treatment tank configured to receive the liquid ejected from the liquid ejection port, the treatment tank allowing an endoscope to be placed inside; a liquid sampling port provided in a connection portion; and a relief cap including a cover configured to cover the liquid sampling port, a fitting portion configured to make the cover be detachably attachable to the connection portion, and an opening/closing portion configured to, if an inner pressure of the liquid supply nozzle exceeds a predetermined value, eject the liquid in a direction with gravity.

### CROSS REFERENCE TO RELATED APPLICATION

This application is a continuation application of PCT/JP2016/059917 filed on Mar. 28, 2016 and claims benefit of Japanese Application No. 2015-172204 filed in Japan on Sep. 1, 2015, the entire contents of which are incorporated herein by this reference.

### BACKGROUND OF THE INVENTION

1. Field of the Invention

The present invention relates to an endoscope reprocessor including a liquid sampling port covered with a cover and provided in a liquid supply nozzle configured to supply a liquid from a liquid ejection port to a treatment tank.

2. Description of the Background Art

Chemical liquid reprocessing of an endoscope using an endoscope reprocessor is performed by supplying a liquid such as a chemical liquid from a liquid ejection port of a liquid supply nozzle to a treatment tank in which the endoscope is placed. The chemical liquid may be a disinfectant liquid.

Also, in order to perform reliable chemical liquid reprocessing of an endoscope, management of a concentration of the chemical liquid is important, and thus, the concentration of the chemical liquid is checked, for example, in each step of the chemical liquid reprocessing.

Japanese Patent No. 5253682 discloses a configuration of an endoscope cleaning/disinfecting apparatus in which a liquid sampling port that can be opened/closed by a lid body is provided in a liquid supply nozzle to enable an effect of a chemical liquid to be confirmed by opening the lid body and then sampling the chemical liquid via the liquid sampling port.

### SUMMARY OF THE INVENTION

An endoscope reprocessor according to an aspect of the present invention includes: a liquid supply nozzle including a lifting conduit configured to feed a liquid in a direction against gravity, and a turndown conduit connected to the lifting conduit via a connection portion, the turndown conduit being configured to change a travel direction of a flow passage in the lifting conduit; a liquid ejection port provided in the turndown conduit; a treatment tank configured to receive the liquid ejected from the liquid ejection port, the treatment tank allowing an endoscope to be placed inside; a liquid sampling port provided in the connection portion; and a relief cap including a cover configured to cover the liquid sampling port, a fitting portion configured to make the cover be detachably attachable to the connection portion, and an opening/closing portion configured to, if an inner pressure of the liquid supply nozzle exceeds a predetermined value, eject the liquid in a direction with gravity.

### BRIEF DESCRIPTION OF THE DRAWINGS

FIG. 1 is a diagram illustrating an overview of a major part of an endoscope reprocessor according to the present embodiment;

FIG. 2 is a diagram schematically illustrating a state in which a hose communicating with a collection container is connected to a liquid ejection port of a turndown conduit in a liquid supply nozzle of the endoscope reprocessor in FIG. 1;

FIG. 3 is a cross-sectional view of a part of the liquid supply nozzle and a relief cap surrounded by line III in FIG. 1;

FIG. 4 is a cross-sectional view of the part of the liquid supply nozzle and the relief cap, which schematically illustrates operation of an opening/closing portion in a case where an inner pressure of the liquid supply nozzle in FIG. 3 exceeds a predetermined value;

FIG. 5 is a cross-sectional view of a part of a liquid supply nozzle and a relief cap, which illustrates a modification of a shape of an inner lid of the relief cap in FIG. 3;

FIG. 6 is a perspective view of a liquid supply nozzle and a relief cap, which illustrates a modification of a fitting portion of the relief cap in FIG. 3;

FIG. 7 is a cross-sectional view of a part of a liquid supply nozzle and a relief cap, which illustrates a modification of an inner structure of the relief cap in FIG. 6; and

FIG. 8 is a perspective view illustrating an example of an endoscope cleaning/disinfecting apparatus in a state in which a top cover is opened and an endoscope can be received in a treatment tank.

### DETAILED DESCRIPTION OF THE PREFERRED EMBODIMENT(S)

An embodiment of the present invention will be described below with reference to the drawings. It should be noted that the drawings are schematic ones and, e.g., a relationship between a thickness and a width of each member and ratios in thickness among the respective members are different from actual ones, and it should be understood that parts that are different in dimensional relationship and ratio depending on the drawings are included in the drawings.

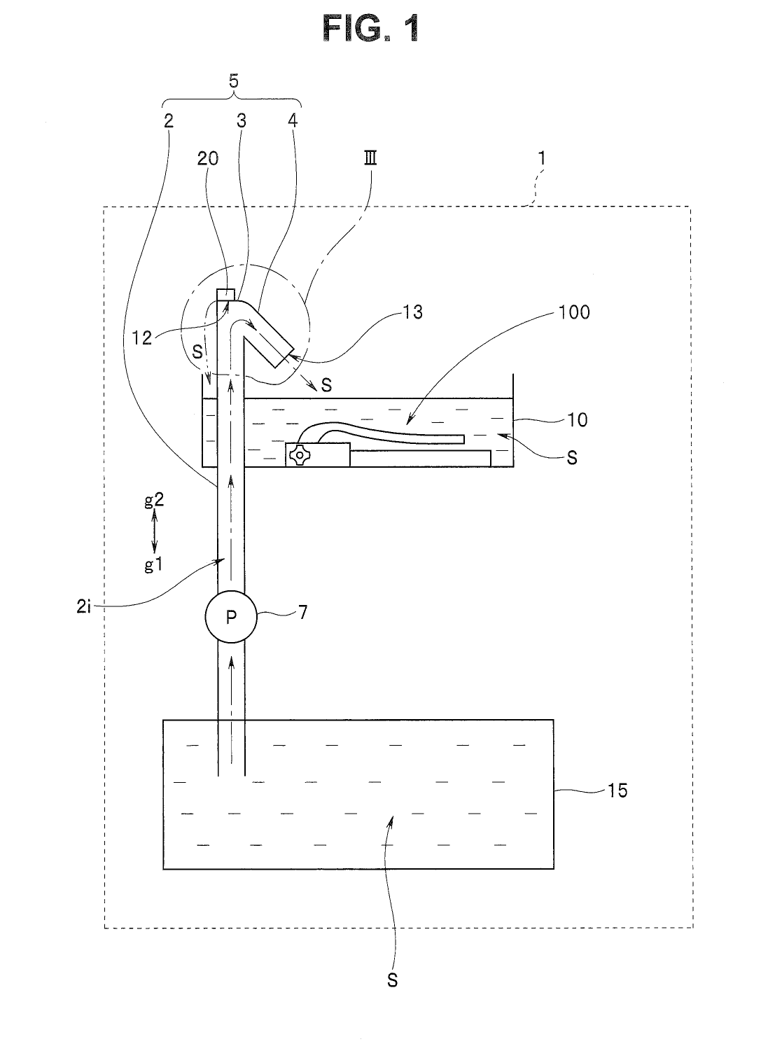

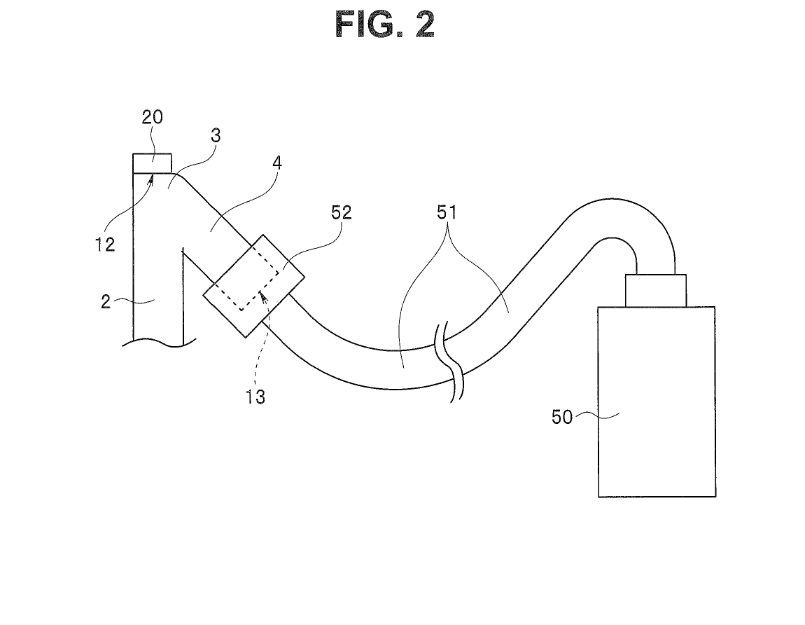

FIG. 1 is a diagram illustrating an overview of a configuration of a major part of an endoscope reprocessor according to the present embodiment, and FIG. 2 is a diagram schematically illustrating a state in which a hose communicating with a collection container is connected to a liquid ejection port of a turndown conduit in a liquid supply nozzle of the endoscope reprocessor in FIG. 1.

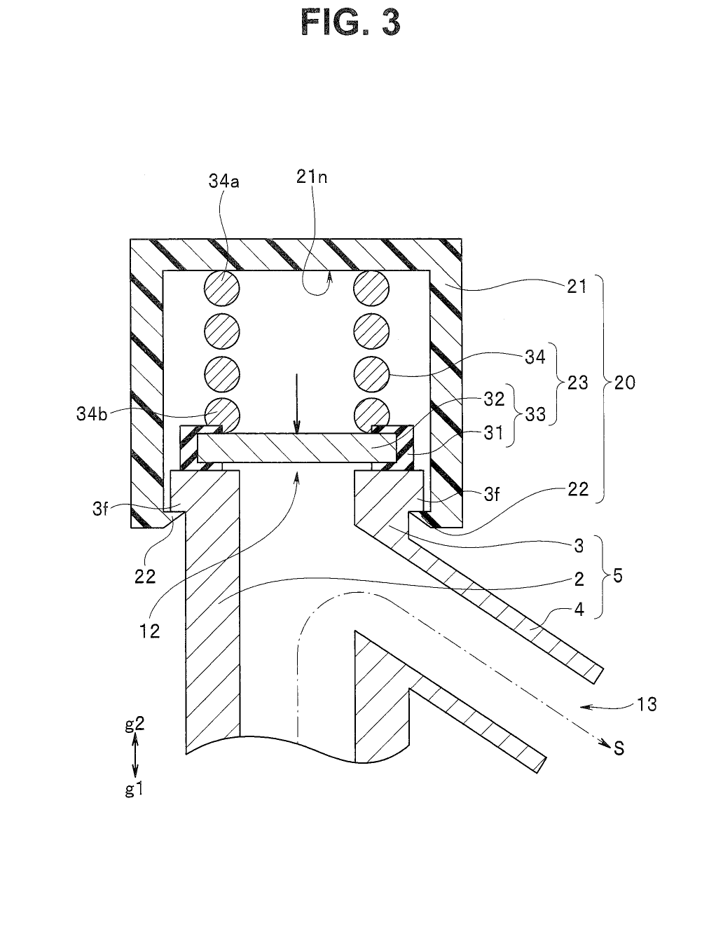

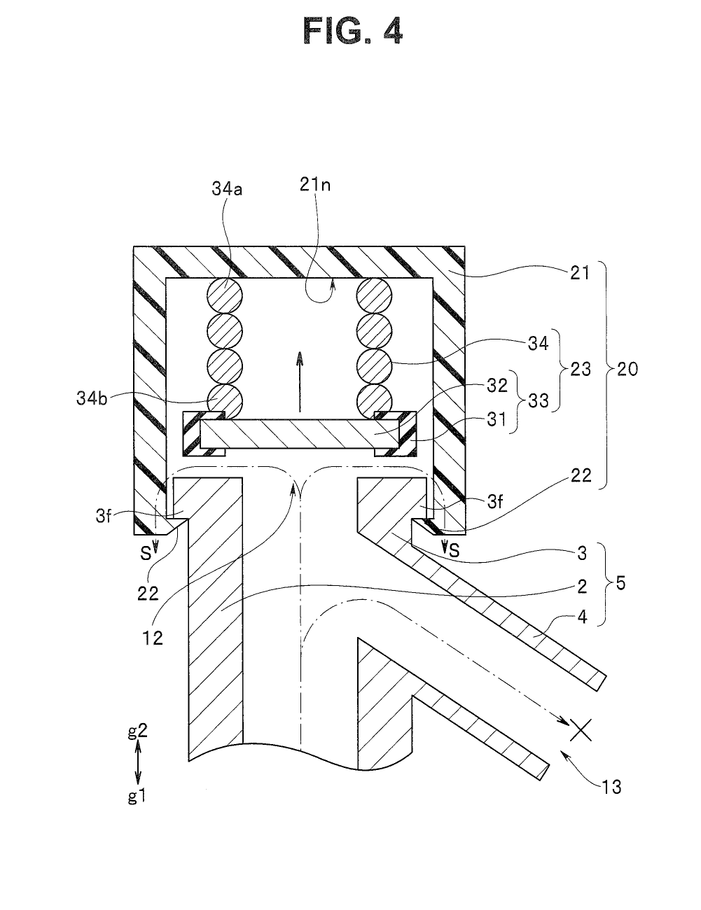

Also, FIG. 3 is a cross-sectional view of a part of the liquid supply nozzle and relief cap surrounded by line III in FIG. 1, and FIG. 4 is a cross-sectional view of the part of the liquid supply nozzle and the relief cap, which schematically illustrates operation of an opening/closing portion in a case where an inner pressure of the liquid supply nozzle in FIG. 3 exceeds a predetermined value.

As illustrated in FIG. 1, the endoscope reprocessor 1 includes a liquid tank 15 configured to store a liquid S, and a treatment tank 10 at a position that is higher than the liquid tank 15 in a direction against gravity g2.

The liquid S may be, but not limited to, a chemical liquid, for example, a cleaning liquid, a disinfectant liquid or a sterile liquid.

Also, the liquid tank 15 is connected to a liquid supply nozzle 5. A major part of the liquid supply nozzle 5 includes a lifting conduit 2, a connection portion 3 and a turndown conduit 4.

The lifting conduit 2 is connected to the liquid tank 15, and a liquid supply member 7 is provided at a position partway of the lifting conduit 2.

With driving of the liquid supply member 7, the lifting conduit 2 feeds the liquid S in the liquid tank 15 in the direction against gravity g2.

The liquid supply member 7 may be a pump, but is not limited to a pump and may be, for example, one configured to contract the liquid tank 15 to push the liquid S out or one configured to pressurize the inside of the liquid tank 15.

The turndown conduit 4 is connected to the lifting conduit 2 via the connection portion 3, and is configured to change a travel direction of a flow passage 2i in the lifting conduit 2 from the direction against gravity g2 to a direction toward the treatment tank 10.

Also, in the turndown conduit 4, a liquid ejection port 13 configured to open toward the treatment tank 10 is formed. Therefore, the liquid S ejected from the liquid ejection port 13 is supplied to the treatment tank 10.

In other words, upon the liquid supply member 7 being driven, the liquid S in the liquid tank 15 is fed through the flow passage 2i of the lifting conduit 2 along the direction against gravity g2, and a travel direction of the liquid S is changed in the turndown conduit 4 via the connection portion 3, and the liquid S is ejected to the treatment tank 10 from the liquid ejection port 13.

The treatment tank 10 allows an endoscope 100 to be reprocessed with the liquid S to be placed inside, and receives the liquid S ejected from the liquid ejection port 13.

Also, as illustrated in FIG. 2, a connector 52 provided at an end portion of a hose 51 configured to communicate with a collection container 50 is connectable to the liquid ejection port 13. The connector 52 is connected to the liquid ejection port 13 when the liquid S in the liquid tank 15 is collected.

In this state, upon the liquid supply member 7 being driven, the liquid S in the liquid tank 15 is fed through the flow passage 2i of the lifting conduit 2 along the direction against gravity g2, the travel direction of the liquid S is changed by the turndown conduit 4 via the connection portion 3, and the liquid S then enters the hose 51 via the liquid ejection port 13 and is collected into the collection container 50.

Referring back to FIG. 1, a liquid sampling port 12 is provided in the connection portion 3 of the liquid supply nozzle 5. Also, the liquid sampling port 12 is occluded by a relief cap 20.

As illustrated in FIG. 3, a major part of the relief cap 20 includes a cover 21, fitting portions 22 and an opening/closing portion 23.

The cover 21 is configured to cover the liquid sampling port 12, and includes a chemical-tolerant material, for example, rubber or plastic.

Each fitting portion 22 is configured to make the cover 21 be detachably attachable to the connection portion 3. More specifically, each fitting portion 22 includes a lug formed integrally with an end portion on the connection portion 3 side of the cover 21, the lug being configured so as to be locked with an outward flange 3f of the connection portion 3.

Each fitting portion 22 may include, e.g. rubber, rather than a lug, and may be formed separately from the cover 21. Also, as described later, the fitting portion 22 may be a slit for receiving a protrusion portion 8 provided on the nozzle side.

Also, the lugs are formed at respective parts in a circumferential direction of an end portion on the connection portion 3 side of the cover 21. Therefore, even if the lugs are locked with the outward flange 3f, gaps are formed at respective parts in the circumferential direction between the fitting portion 22 and the outward flange 3f.

The opening/closing portion 23 is provided inside the cover 21, and is configured to, upon an inner pressure of the liquid supply nozzle 5 exceeding a predetermined value, eject the liquid S to an outer periphery of the liquid supply nozzle 5 in a direction with gravity g1.

More specifically, the opening/closing portion 23 includes an inner lid 33 and a biasing portion 34 each including a chemical-tolerant material.

The inner lid 33 includes a plate material 32 and a watertight member 31, and is configured to occlude the liquid sampling port 12 in a watertight manner

The plate material 32 and the watertight member 31 may be formed integrally. In other words, the plate material 32 itself may include a watertight member. Also, an outer periphery of the inner lid 33 is covered by the cover 21.

The biasing portion 34 includes one end 34a configured to be in abutment with an inner face 21n of the cover 21 and the other end 34b configured to press the inner lid 33 against the liquid sampling port 12 in the direction with gravity g1. For the biasing portion 34, for example, an elastic body can be used. The elastic body may be, for example, a spring or an elastomer.

Although in FIGS. 3 and 4, as an example of the biasing portion 34, a coil spring is illustrated, the biasing portion 34 may be a leaf spring, and furthermore, may include any member other than a spring as long as the member can bias the inner lid 33 of, e.g., rubber toward the liquid sampling port 12.

As illustrated in FIG. 3, a biasing force of the biasing portion 34, the biasing force biasing the inner lid 33 toward the liquid sampling port 12, that is, a spring pressure, is set to be higher than the inner pressure of the liquid supply nozzle 5 when the liquid supply member 7 is driven to supply the liquid S from the liquid tank 15 to the treatment tank 10 via the liquid ejection port 13.

Consequently, when the liquid S is supplied to the treatment tank 10, the water-tightness of the liquid sampling port 12 provided by the inner lid 33 is prevented from being cancelled.

In addition, as illustrated in FIG. 4, the spring pressure is set to be lower than the inner pressure of the liquid supply nozzle 5 when the inner pressure exceeds the predetermined value as a result of, e.g., the hose 51 being buckled. Consequently, the spring pressure is set so that the water-tightness of the liquid sampling port 12 provided by the inner lid 33 is cancelled.

Next, operation of the present embodiment will be described.

First, when the liquid supply member 7 is driven to supply the liquid S from the liquid tank 15 to the treatment tank 10 via the liquid ejection port 13 through the lifting conduit 2, the connection portion 3 and the turndown conduit 4, the spring pressure of the biasing portion 34 is set to be higher than the inner pressure of the liquid supply nozzle 5. Therefore, as illustrated in FIG. 3, the biasing portion 34 presses the inner lid 33 toward the liquid sampling port 12, and thus the liquid sampling port 12 is occluded by the inner lid 33 in a watertight manner. Accordingly, the liquid S is prevented from leaking from the liquid sampling port 12.

As illustrated in FIG. 2, the above can apply to a case where the connector 52 of the hose 51 is connected to the liquid ejection port 13 to collect the liquid S from the liquid tank 15 into the collection container 50 via the lifting conduit 2, the connection portion 3, the turndown conduit 4, the liquid ejection port 13 and the hose 51.

Here, as illustrated in FIG. 4, if the inner pressure of the liquid supply nozzle 5 exceeds the predetermined value as a result of, for example, the hose 51 being buckled at a position partway of the hose 51, as described above, the spring pressure of the biasing portion 34 is set to be lower than the inner pressure of the liquid supply nozzle 5 when the inner pressure of the liquid supply nozzle 5 exceeds the predetermined value. Consequently, before the fitting portion 22 is broken, the inner lid 33 is moved against the biasing of the biasing portion 34 in the direction against gravity g2 by the inner pressure.

As a result, the liquid sampling port 12 is opened, and the liquid S flows into the cover 21 via the liquid sampling port 12. Subsequently, the liquid S is ejected in the direction with gravity g1 via the gaps formed between the fitting portions 22 and the outward flange 3f.

The ejected liquid S, as indicated by an alternate long and two short dashes line in FIG. 1, flows into the treatment tank 10, and then, for example, collected into the liquid tank 15 again.

Also, in this case, the liquid sampling port 12 is opened by the inner lid 33, and thus, the inner pressure of the liquid supply nozzle 5 can be released via the liquid sampling port 12, and thus, the fitting portions 22 are prevented from coming off from the outward flange 3f.

In other words, the fitting portions 22 are prevented from being broken. In brief, the relief cap 20 is prevented from coming off from the liquid sampling port 12.

Furthermore, as described above the cover 21 covers the periphery of the inner lid 33 and the liquid S flowing out from the liquid sampling port 12 by the inner face 21n of the cover 21 is guided in the direction with gravity g1, and thus, the liquid S flowing out from the liquid sampling port 12 is prevented from being splattered around and is collected into the treatment tank 10.

As described above, the present embodiment indicates that the relief cap 20 configured to occlude the liquid sampling port 12 formed in the connection portion 3 includes the opening/closing portion 23 configured to, if the inner pressure of the liquid supply nozzle 5 exceeds the predetermined value, eject the liquid S in the direction with gravity g1.

More specifically, the present embodiment indicates that if the inner pressure of the liquid supply nozzle 5 exceeds the predetermined value, the liquid sampling port 12 is opened as a result of the inner lid 33 being moved against the biasing of the biasing portion 34 in the direction against gravity g2 by the inner pressure, before the fitting portions 22 are broken.

Accordingly, even if the inner pressure of the liquid supply nozzle 5 exceeds the predetermined value as a result of, e.g., the hose 51 connected to the liquid ejection port 13 being buckled, the liquid sampling port 12 is opened and the inner pressure can be released, preventing the relief cap 20 from coming off from the liquid sampling port 12. In other words, the fitting portions 22 are prevented from being broken and thus, the cover 21 is prevented from coming off from the liquid sampling port 12.

Also, even if the liquid sampling port 12 is opened, since the cover 21 covers the outer periphery of the inner lid 33 and the liquid S is guided by the inner face 21n of the cover 21 and ejected in the direction with gravity g1 and flows into the treatment tank 10, and thus the liquid S is prevented from being splattered around.

Furthermore, the liquid supply member 7 can be prevented from failing as a result of a load being imposed on the liquid supply member 7 when the inner pressure of the liquid supply nozzle 5 exceeds the predetermined value.

According to the above, an endoscope reprocessor 1 including a configuration that can prevent the cover 21 covering the liquid sampling port 12 from coming off even if the inner pressure of the liquid supply nozzle 5 increases can be provided.

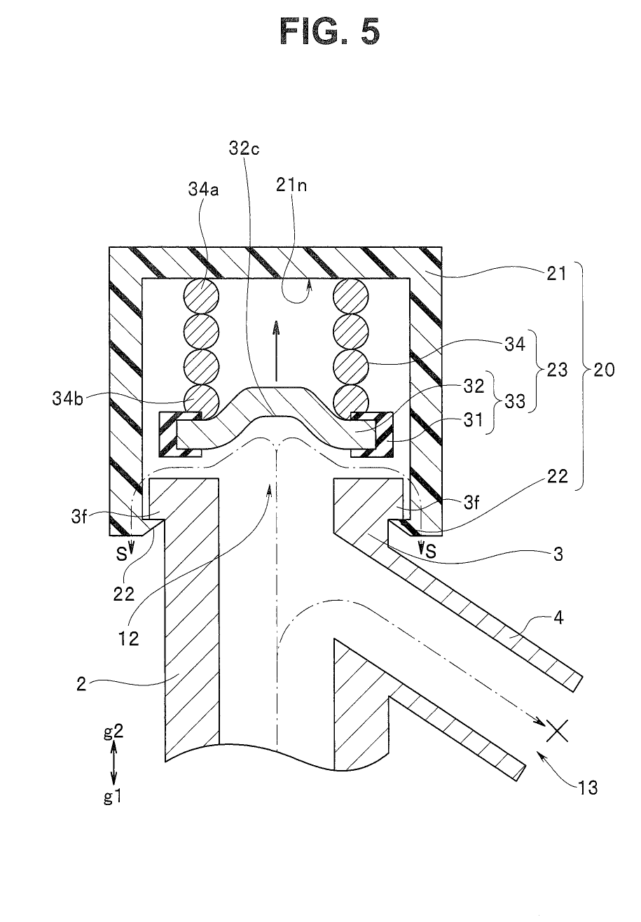

A modification will be indicated below with reference to FIG. 5. FIG. 5 is a cross-sectional view of a part of a liquid supply nozzle and a relief cap, which illustrates a modification of a shape of the inner lid of the relief cap in FIG. 3.

As illustrated in FIG. 5, an inner lid 33 may be formed in a shape in which a plate material 32 includes a recess portion 32c including a recess facing the liquid sampling port 12.

More specifically, the plate material 32 of the inner lid 33 may be formed in a shape protruding in the direction g2 because of a surface of the plate material 32, the surface facing the liquid sampling port 12, being recessed in a direction against gravity g2.

Consequently, a liquid S flowing out from the liquid sampling port 12 can actively be guided in a direction with gravity g1 by the recess portion 32c.

Also, even if the cover 21 does not cover the outer periphery of the inner lid 33, the recess portion 32c guides the liquid S flowing out from the liquid sampling port 12 so as to prevent the liquid S from being splattered around.

Other effects are similar to the effects of the present embodiment described above.

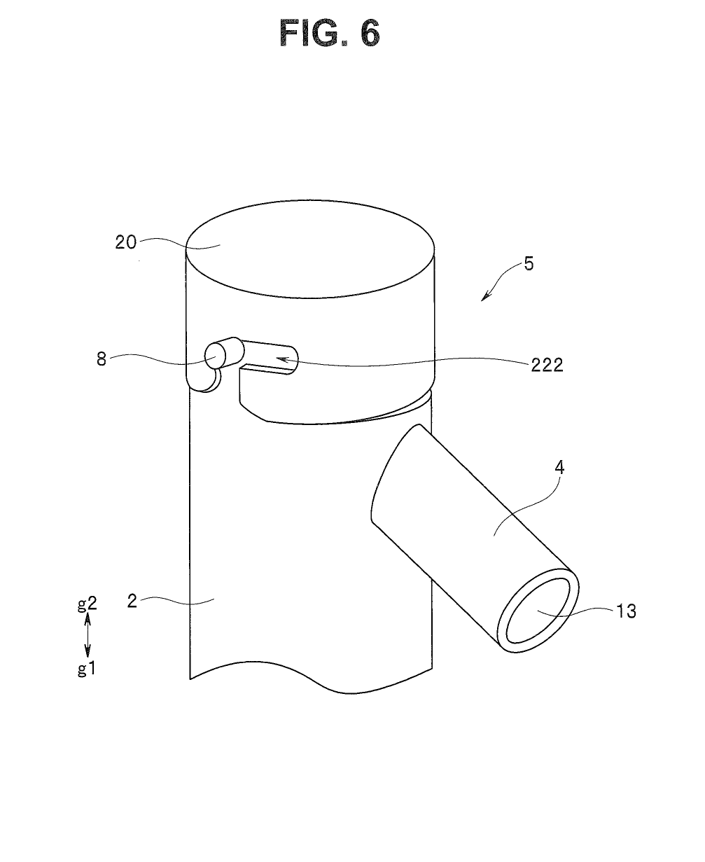

As illustrated in FIG. 6, it is possible that: a protrusion portion 8 is provided on the nozzle side; and a slit-like fitting portion 222 is provided in the relief cap 20. The number of protrusion portions 8 and the number of fitting portions 222 are not specifically limited, and a single protrusion portion 8 or a plurality of protrusion portions 8, and a single fitting portion 222 or a plurality of fitting portions 222 may be provided, respectively.

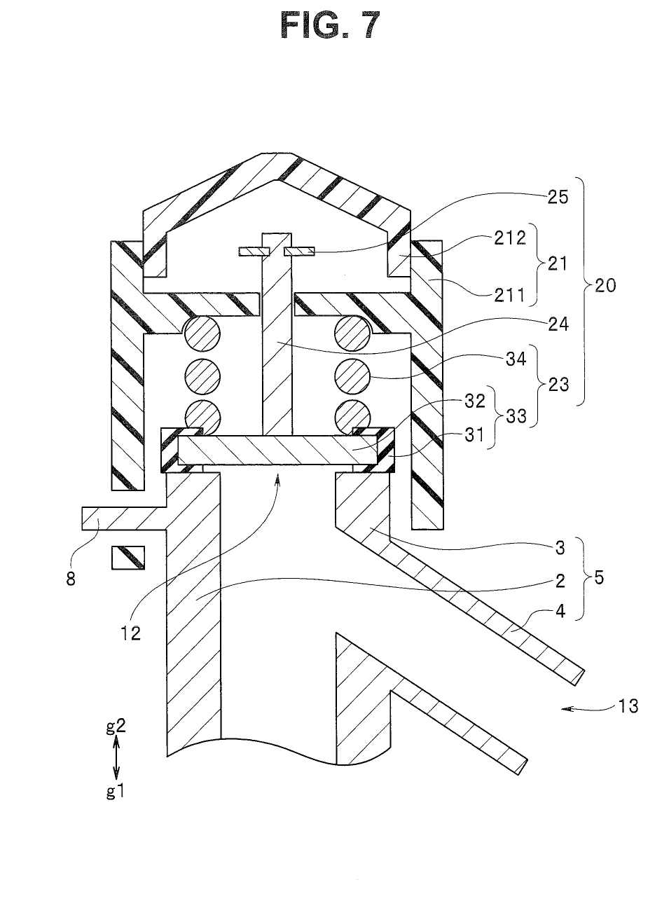

As illustrated in FIG. 7, it is possible that a cover 21 is divided in a first cover 211 and a second cover 212; and a plate material 32 is connected to the first cover 211 by a strut 24 and a stopper 25.

As in FIG. 7, disposition of a biasing portion 34 between the plate material 32 and the first cover 211 around an outer periphery of the strut 24 enables the biasing portion 34 to function without the biasing portion 34 being fixed to the cover 21. As a result, work for fixing the biasing portion 34 can be omitted, enabling enhancement in yield.

Since the strut 24 can advance/retract relative to the first cover 211, the first cover 211 and the second cover 212 are desirably connected in a watertight manner Although not specifically limited, an E-shaped ring can be used as the stopper 25.

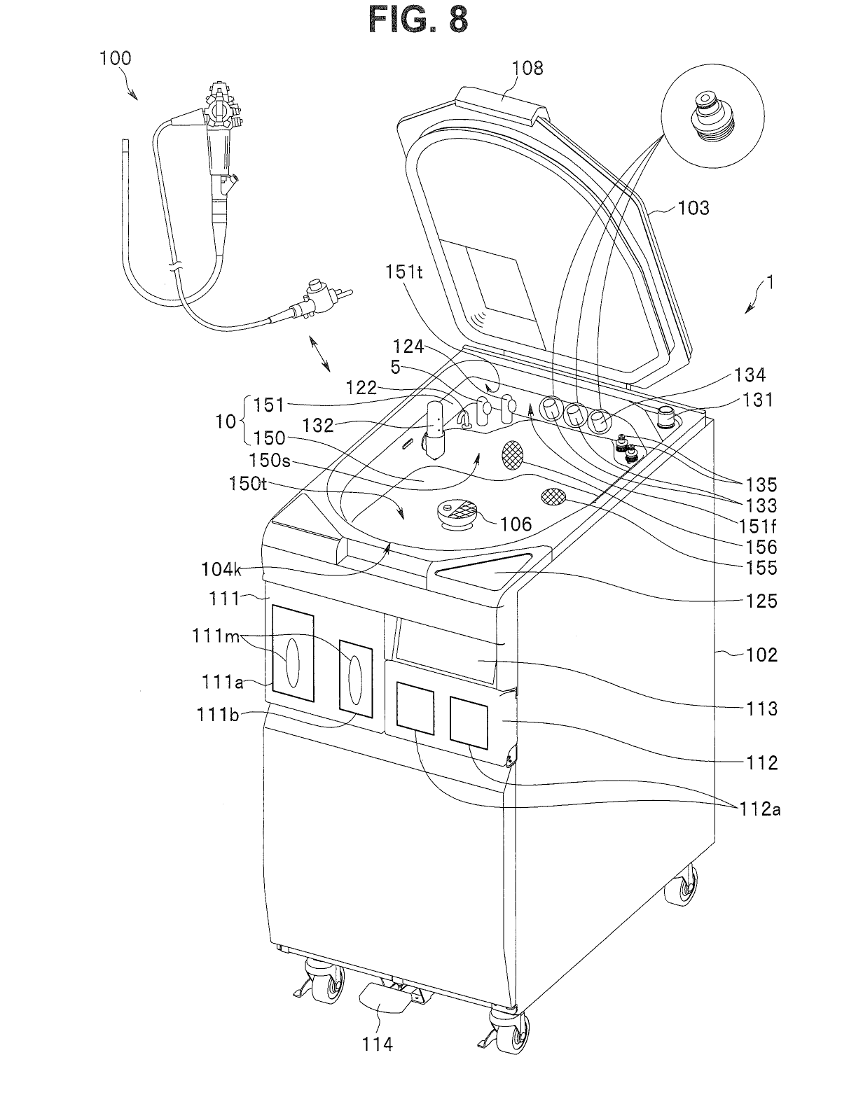

Next, an example of the endoscope reprocessor 1, for example, an endoscope cleaning/disinfecting apparatus, according to the present embodiment will be described with reference to FIG. 8.

FIG. 8 is a perspective view of an example of an endoscope cleaning/disinfecting apparatus in a state in which a top cover is opened and an endoscope can be received in a treatment tank. Hereinafter, reference numeral 1 is provided also to the endoscope cleaning/disinfecting apparatus.

As illustrated in the figure, the endoscope cleaning/disinfecting apparatus 1 is an apparatus for cleaning/disinfecting a used endoscope 100, and a major part of the endoscope cleaning/disinfecting apparatus 1 includes an apparatus body 102, and a top cover 103 connected to an upper portion of the apparatus body 102 so as to be openable/closable, via, for example, a non-illustrated hinge.

In a state in which the top cover 103 is closed on the apparatus body 102, the apparatus body 102 and the top cover 103 are fixed via, for example, a latch 108 arranged at a position at which the apparatus body 102 and the top cover 103 face each other.

In, for example, an upper portion of a left half portion in a front face in the figure, which is close to an operator of the apparatus body 102, a detergent/alcohol tray 111 is arranged in such a manner that the detergent/alcohol tray 111 can be drawn forward of the apparatus body 102.

In the detergent/alcohol tray 111, a detergent tank 111a configured to store a cleaning agent to be used for cleaning the endoscope 100 and an alcohol tank 111b configured to store alcohol to be used for drying the cleaned/disinfected endoscope 100 are housed, and since the detergent/alcohol tray 111 can be drawn out, the respective tanks 111a, 111b can be replenished with the respective liquids in a predetermined manner.

In the detergent/alcohol tray 111, two window portions 111m are provided, and remaining amounts of the cleaning agent and the alcohol stored in the respective tanks 111a, 111b can be recognized by the operator through the window portions 111m.

Also, in, for example, an upper portion of a right half portion in the front face of the apparatus body 102, a cassette tray 112 is arranged in such a manner that the cassette tray 112 can be drawn forward of the apparatus body 102. In the cassette tray 112, a chemical liquid bottle 112a in which a disinfectant liquid of, for example, a peracetic acid, to be used for disinfecting the endoscope 100 is stored is housed, and since the cassette tray 112 can be drawn out, the chemical liquid bottle 112a can be set in a predetermined manner.

Furthermore, in a part above the cassette tray 112 in the front face of the apparatus body 102, a sub operation panel 113 in which cleaning/disinfecting time period is displayed and instruction buttons for disinfectant liquid warming or the like are arranged is arranged.

Also, at a lower portion in the front face in the figure of the apparatus body 102, a pedal switch 114 configured to open the top cover 103 closed on the upper portion of the apparatus body 102, upward of the apparatus body 102 by means of stepping operation of the operator is arranged.

Also, at, for example, a position close to an end on the front side, which is close to the operator, in an upper face of the apparatus body 102, a main operation panel 125 in which setting switches and the like for the apparatus body 102 such as a cleaning/disinfecting operation start switch and a cleaning/disinfecting mode selection switch are arranged is provided.

Also, on the back side opposed to the front face close to the operator in the upper face of the apparatus body 102, a water supply hose connection port 131 configured to supply tap water to supply the apparatus body 102, a non-illustrated water supply hose connected to a tap water faucet being connected to the water supply hose connection port 131, is arranged. Also, a mesh filter configured to filtrate tap water may be arranged in the water supply hose connection port 131.

Furthermore, in a roughly center portion in the upper face of the apparatus body 102, a treatment tank 10 configured to receive the endoscope 100, the treatment tank 10 including an endoscope receiving opening configured to be opened/closed by the top cover 103, is provided. The treatment tank 10 includes, for example, a tank body 150, and a terrace portion 151 provided around the tank body 150 so as to be continuous with an outer peripheral edge of the endoscope receiving port of the tank body 150.

When the used endoscope 100 is cleaned/disinfected, the tank body 150 can receive the endoscope 100, and in a bottom face 150t, which is a surface inside the tank body 150, a first drain port 155 for drainage of, e.g., a cleaning liquid, water, alcohol or a disinfectant liquid supplied in the tank body 150, from the tank body 150.

Also, at an arbitrary position in a circumferential side face 150s, which is a surface inside the tank body 150, a circulation port 156 configured to supply, e.g., a cleaning liquid, water or a disinfectant liquid supplied in the tank body 150, to each of conduits included inside the endoscope 100 or supply the aforementioned liquid again to the tank body 150 from a water supply/circulation nozzle 124 via, e.g., a mesh filter is provided. A mesh filter configured to filtrate, e.g., a cleaning liquid may be provided in the circulation port.

The above-described circulation port 156 may be provided in the bottom face 150t of the tank body 150. If the circulation port 156 is provided in the bottom face 150t of the tank body 150, a timing for supplying, e.g., a cleaning liquid, water or a disinfectant liquid to each of the conduits of the endoscope 100 or supplying the aforementioned liquid again to the tank body 150 can be advanced. Furthermore, the provision of the circulation port 156 in the bottom face provides an advantage in that the operator can easily approaches the circulation port 156 when the operator replaces, e.g., the mesh filter provided in the circulation port 156.

At a roughly center portion in the bottom face 150t of the tank body 150 of the treatment tank 10, a cleaning case 106 is arranged.

In the cleaning case 106, buttons and the like such as respective scope switches of the endoscope 100 and removable components provided together with the endoscope 100 are housed. As a result, the respective buttons and the like and the removed components are cleaned and disinfected together with the endoscope 100.

At an arbitrary position in the side face 150s of the tank body 150, a level sensor 132 with a cover, the level sensor 132 being configured to detect a level of a cleaning liquid, water or a disinfectant liquid supplied in the tank body 150, is provided.

In a surface other than the terrace surface 151t of the terrace portion 151, that is, a surface of the terrace portion 151, the surface being parallel to the bottom face 150t of the tank body 150, a detergent nozzle 122 configured to supply a cleaning agent to be diluted to a predetermined concentration by tap water, from the detergent tank 111a to the tank body 150, and a disinfectant liquid nozzle 5, which corresponds to a liquid supply nozzle of the present embodiment for supplying a disinfectant liquid, which is an example of the liquid S, from the liquid tank 15 (see FIG. 1) by means of the liquid supply member 7 (see FIG. 1), are arranged.

Furthermore, in the surface of the terrace portion 151, the surface being parallel to the bottom face 150t of the tank body 150, a water supply/circulation nozzle 124 configured to supply water to the tank body 150 or supply, e.g., a cleaning liquid, water or a disinfectant liquid sucked from the circulation port 156 of the tank body 150, to the tank body 150 again is arranged.

The detergent nozzle 122, the disinfectant liquid nozzle 5 and the water supply/circulation nozzle 124 may be arranged in the terrace surface 151t.

Also, in a surface 151f of the terrace surface 151t of the terrace portion 151 on the side opposed to a position 104k close to the operator, a plurality of, here, two air/water feeding/forceps ports 133 configured to supply, e.g., a cleaning liquid, water, alcohol, a disinfectant liquid or air to the conduits included inside the endoscope 100, a forceps elevation port 134, and a leakage detection port 135 are arranged.

Note that the configuration of the endoscope cleaning/disinfecting apparatus described above is definitely an example, and the present invention is not limited to the configuration in FIG. 8.

Also, it should be understood that an endoscope reprocessor is not limited to an endoscope cleaning/disinfecting apparatus.

What is claimed is:

1. An endoscope reprocessor comprising:
a liquid supply nozzle including a lifting conduit configured to feed a liquid in a direction against gravity, and a turndown conduit connected to the lifting conduit via a connection portion, the turndown conduit being configured to change a travel direction of a flow passage in the lifting conduit;
a liquid ejection port provided in the turndown conduit;
a treatment tank configured to receive the liquid ejected from the liquid ejection port, the treatment tank allowing an endoscope to be placed inside;
a liquid sampling port provided in the connection portion; and
a relief cap including a cover configured to cover the liquid sampling port, a fitting portion configured to make the cover be detachably attachable to the connection portion, and an opening/closing portion configured to, if an inner pressure of the liquid supply nozzle exceeds a predetermined value, eject the liquid in a direction with gravity.

2. The endoscope reprocessor according to claim 1, wherein the opening/closing portion includes:
an inner lid disposed in an inner portion of the cover, the inner lid being configured to occlude the liquid sampling port in a watertight manner; and
a biasing portion including an end configured to be in abutment with an inner face of the cover, and another end configured to press the inner lid against the liquid sampling port in the direction with gravity.

3. The endoscope reprocessor according to claim 2, wherein if the inner pressure of the liquid supply nozzle exceeds the predetermined value, the liquid sampling port is opened as a result of the inner lid being moved against the biasing of the biasing portion in the direction against gravity by the inner pressure, and the liquid is ejected in the direction with gravity via the liquid sampling port and the fitting portion.

4. The endoscope reprocessor according to claim 2, wherein the inner lid includes a recess portion including a recess facing the liquid sampling port.
